(19) United States
(12) Patent Application Publication
(10) Pub. No.: US 2024/0366883 A1
(43) Pub. Date: Nov. 7, 2024

(54) Needle Shield Remover Assembly for Wearable Injector

(71) Applicant: Becton, Dickinson and Company, Franklin Lakes, NJ (US)

(72) Inventor: Trivikrama Bhanoji Pala, Parsippany, NJ (US)

(21) Appl. No.: 18/580,271

(22) PCT Filed: Jul. 18, 2022
(86) PCT No.: PCT/US22/37425

§ 371 Date: Jan. 18, 2024

Related U.S. Application Data
(60) Provisional application No. 63/223,181, filed on Jul. 19, 2021.

(51) Int. Cl. A61M 5/32; A61M 5/158

(52) U.S. Cl. CPC A61M 5/3243; A61M 5/158

(57) ABSTRACT

A needle shield remover assembly for a wearable injector includes a needle shield having a body defining an opening configured to receive a patient needle of the wearable injector, with the body of the needle shield having an extension portion having a top surface and a bottom surface positioned opposite the top surface, and a needle shield remover having a first portion and a second portion. The needle shield remover has an unassembled position where the first portion is spaced from the second portion and an assembled position where the first portion is connected to the second portion, with the first portion and the second portion of the needle shield remover defining a needle shield surface configured to engage the top surface of the extension portion of the needle shield when the needle shield remover is in the assembled position.

### CROSS-REFERENCE TO RELATED APPLICATION

The present application claims priority to U.S. Provisional Application Ser. No. 63/223,181, entitled “Needle Shield Removed Assembly for Wearable Injector”, filed Jul. 19, 2021, the entire disclosure of which is hereby incorporated by reference in its' entirety.

### BACKGROUND OF THE INVENTION

### Field of the Invention

The present disclosure relates generally to a needle shield remover assembly for a wearable injector.

### Description of Related Art

Various types of automatic injection or drug delivery devices have been developed to allow drug solutions and other liquid therapeutic preparations to be administered by untrained personnel or to be self-injected. Generally, these devices include a reservoir that is pre-filled with the liquid therapeutic preparation, and some type of automatic needle-injection mechanism that can be triggered by the user. When the volume of fluid or drug to be administered is generally below a certain volume, such as 1 mL, an auto-injector is typically used, which typically has an injection time of about 10 to 15 seconds. When the volume of fluid or drug to be administered is above 1 mL, the injection time generally becomes longer resulting in difficulties for the patient to maintain contact between the device and the target area of the patient's skin. Further, as the volume of drug to be administered becomes larger, increasing the time period for injection becomes desirable. The traditional method for a drug to be injected slowly into a patient is to initiate an IV and inject the drug into the patient's body slowly. Such a procedure is typically performed in a hospital or outpatient setting.

Certain devices allow for self-injection in a home setting and are capable of gradually injecting a liquid therapeutic preparation into the skin of a patient. In some cases, these devices are small enough (both in height and in overall size) to allow them to be “worn” by a patient while the liquid therapeutic preparation is being infused into the patient. These devices typically include a pump or other type of discharge mechanism to force the liquid therapeutic preparation to flow out of a reservoir and into the injection needle. Such devices also typically include a valve or flow control mechanism to cause the liquid therapeutic preparation to begin to flow at the proper time and a triggering mechanism to initiate the injection.

Referring to FIG. 16, in order to maintain sterility and to prevent accidental needle sticks, the needle of a drug delivery device is covered by a needle shield prior to use. The needle shield may be removed by grasping the needle shield or by grasping a needle shield remover that removes the needle shield when the needle shield remover is separated from the device. In certain cases, due to the connection between the needle shield and the needle shield remover, the needle shield can fail to separate from the device when the needle shield remover is separated from the device.

### SUMMARY OF THE INVENTION

In one aspect or embodiment, a needle shield remover assembly for a wearable injector includes a needle shield having a body defining an opening configured to receive a needle of the wearable injector, with the body of the needle shield including an extension portion having a top surface and a bottom surface positioned opposite the top surface, and a needle shield remover including a first portion and a second portion, with the needle shield remover having an unassembled position where the first portion is spaced from the second portion and an assembled position where the first portion is connected to the second portion. The first portion and the second portion of the needle shield remover define a needle shield surface configured to engage the top surface of the extension portion of the needle shield when the needle shield remover is in the assembled position.

The body of the needle shield may have a first end and a second end positioned opposite the first end, with the opening of the body positioned at the first end and the extension portion positioned at the second end. The extension portion may include a disc-shaped member. The extension portion may include a cylindrical member, with the disc-shaped member defining the second end of the body of the needle shield. The body of the needle shield may include a protrusion having a tapered surface and a planar surface, the tapered surface positioned closer to the second end of the body than the planar surface. The needle shield remover may include a plurality of needle shield engagement members, with the plurality of needle shield engagement members configured to be biased radially outward when engaged with the tapered surface of the body of the needle shield, and with the plurality of needle shield engagement members configured to engage the planar surface of the needle shield after the needle shield remover is connected to the needle shield. The first portion and the second portion of the needle shield remover may each include at least one of the plurality of needle shield engagement members.

The first portion of the needle shield remover may be connected to the second portion of the needle shield remover via a connector. The connector may include a connector recess positioned on one of the first portion and the second portion and a connector member positioned on the other of the first portion and the second portion. The connector recess and the connector member may form a snap fit connection.

The second portion of the needle shield remover may include a device connector configured to secure the second portion of the needle shield remover to the wearable injector. The first portion of the needle shield remover may include a handle. The second portion of the needle shield remover may include a first half moveable relative to a second half, where one of the first half and the second half includes a cover configured to cover the needle shield after the needle shield remover is connected to the needle shield. The first half of the second portion of the needle shield remover may be connected to the second half of the second portion of the needle shield remover via a hinge.

In a further aspect or embodiment, a wearable injector includes a housing having a top surface and a bottom surface, a container received within the housing, with the container configured to receive a medicament and including a stopper configured to move within the container, a drive assembly configured to move the stopper, a needle having a retracted position where the needle is positioned within the housing and an extended position where a portion of the needle protrudes from the housing, and a needle shield remover assembly of any of the aspects or embodiments discussed above. The needle shield receives at least a portion of the needle, and the needle shield remover is in the assembled position.

### BRIEF DESCRIPTION OF THE DRAWINGS

The above-mentioned and other features and advantages of this disclosure, and the manner of attaining them, will become more apparent and the disclosure itself will be better understood by reference to the following descriptions of embodiments of the disclosure taken in conjunction with the accompanying drawings, wherein:

FIG. 1 is a perspective view of a wearable injector according to one aspect of the present invention.

FIG. 2 is a perspective, cross-sectional view of the wearable injector of FIG. 1 according to one aspect of the present invention.

FIG. 3 is a front, cross-sectional view of the wearable injector of FIG. 1 according to one aspect of the present invention.

FIG. 4 is a top view of the wearable injector of FIG. 1 according to one aspect of the present invention, showing a top portion of the housing removed and the wearable injector in a pre-use position.

FIG. 5 is a top, cross-sectional view of the wearable injector of FIG. 1 according to one aspect of the present invention, showing the wearable injector in a pre-use position.

FIG. 6 is a front, cross-sectional view of the wearable injector of FIG. 1 according to one aspect of the present invention, showing the wearable injector in a pre-use position.

FIG. 7 is a top view of the wearable injector of FIG. 1 according to one aspect of the present invention, showing a top portion of the housing removed and the wearable injector in an initial actuation position.

FIG. 8 is a top, cross-sectional view of the wearable injector of FIG. 1 according to one aspect of the present invention, showing the wearable injector in an initial actuation position.

FIG. 9 is a front, cross-sectional view of the wearable injector of FIG. 1 according to one aspect of the present invention, showing the wearable injector in an initial actuation position.

FIG. 10 is a top view of the wearable injector of FIG. 1 according to one aspect of the present invention, showing a top portion of the housing removed and the wearable injector in a use position.

FIG. 11 is a top, cross-sectional view of the wearable injector of FIG. 1 according to one aspect of the present invention, showing the wearable injector in a use position.

FIG. 12 is a front, cross-sectional view of the wearable injector of FIG. 1 according to one aspect of the present invention, showing the wearable injector in a use position.

FIG. 13 is a top view of the wearable injector of FIG. 1 according to one aspect of the present invention, showing a top portion of the housing removed and the wearable injector in a post-use position.

FIG. 14 is a top, cross-sectional view of the wearable injector of FIG. 1 according to one aspect of the present invention, showing the wearable injector in a post-use position.

FIG. 15 is a front, cross-sectional view of the wearable injector of FIG. 1 according to one aspect of the present invention, showing the wearable injector in a post-use position.

FIG. 16 is a partial cross-sectional view of a prior art needle shield remover assembly.

FIG. 17 is a partial cross-sectional view of a needle shield remover assembly according to one aspect or embodiment of the present application, showing the needle shield remover assembly connected to the wearable injector.

FIG. 18 is a perspective view of a needle shield according to one aspect or embodiment of the present application.

FIG. 19 is a perspective view of the needle shield remover assembly of FIG. 16, showing an assembled position of the needle shield remover.

FIG. 20 is a perspective view of the needle shield remover assembly of FIG. 16, showing an unassembled position of the needle shield remover.

FIG. 21 is a perspective view of a second portion of the needle shield remover of FIG. 16.

FIG. 22 is a perspective view of a first portion of the needle shield remover of FIG. 16.

FIG. 23 is a partial cross-sectional view of the needle shield remover assembly of FIG. 16.

### DETAILED DESCRIPTION

The following description is provided to enable those skilled in the art to make and use the described embodiments contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present invention.

For purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawing figures. However, it is to be understood that the invention may assume various alternative variations, except where expressly specified to the contrary. It is also to be understood that the specific devices illustrated in the attached drawings, and described in the following specification, are simply exemplary embodiments of the invention. Hence, specific dimensions and other physical characteristics related to the embodiments disclosed herein are not to be considered as limiting.

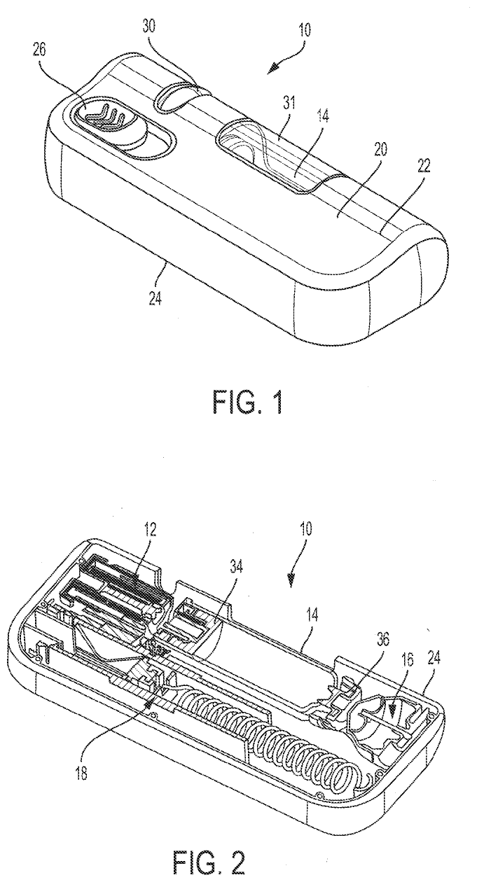

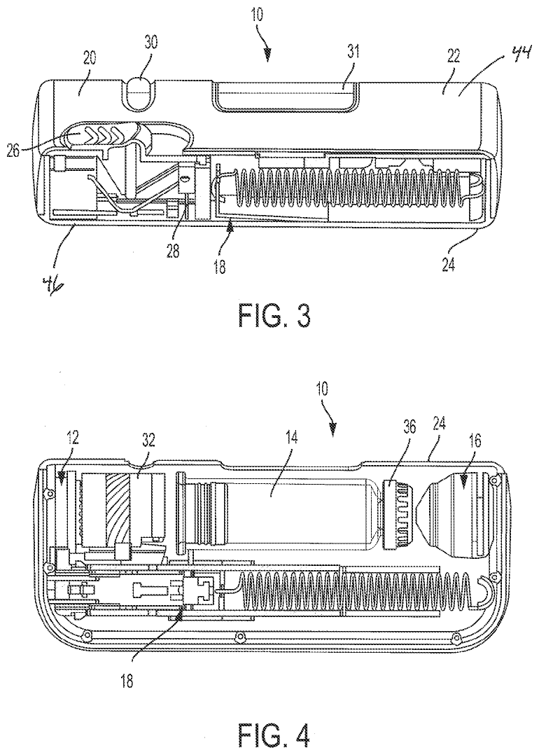

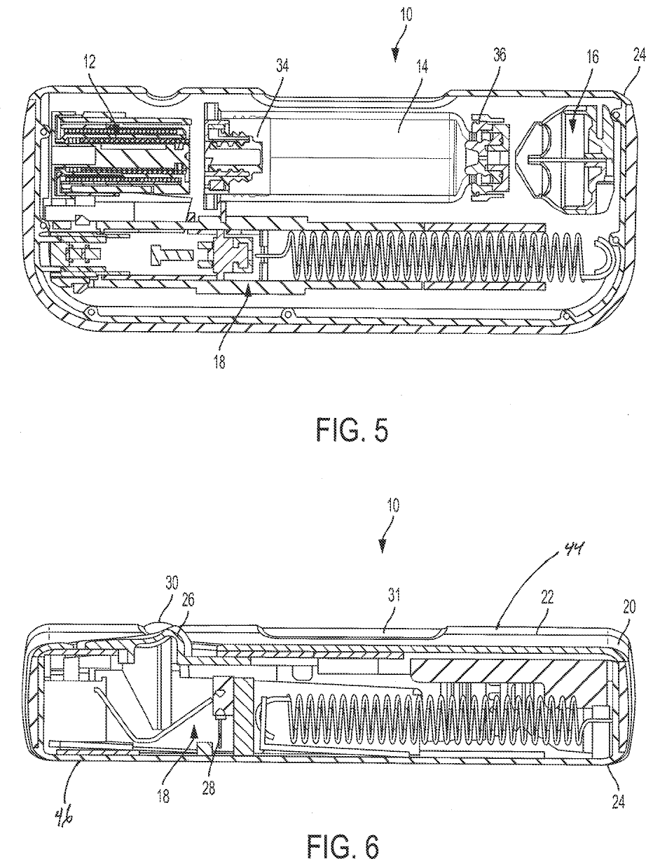

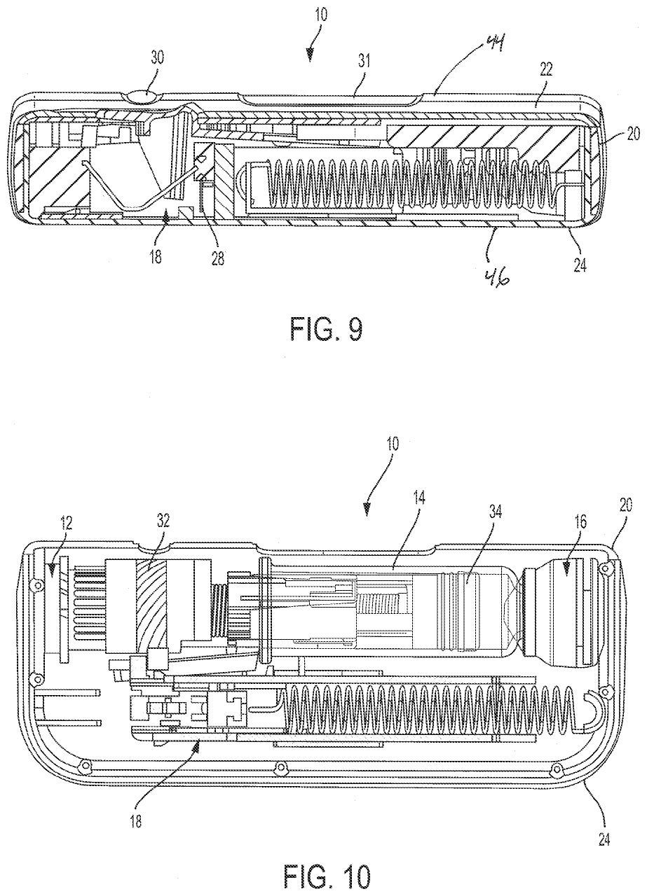

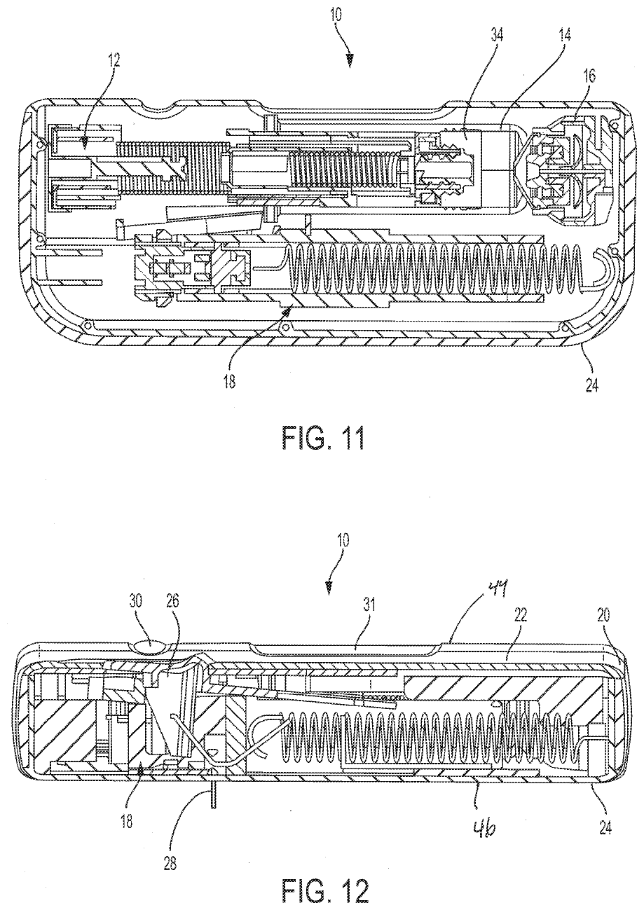

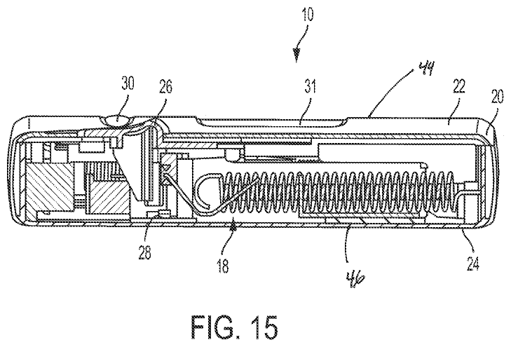

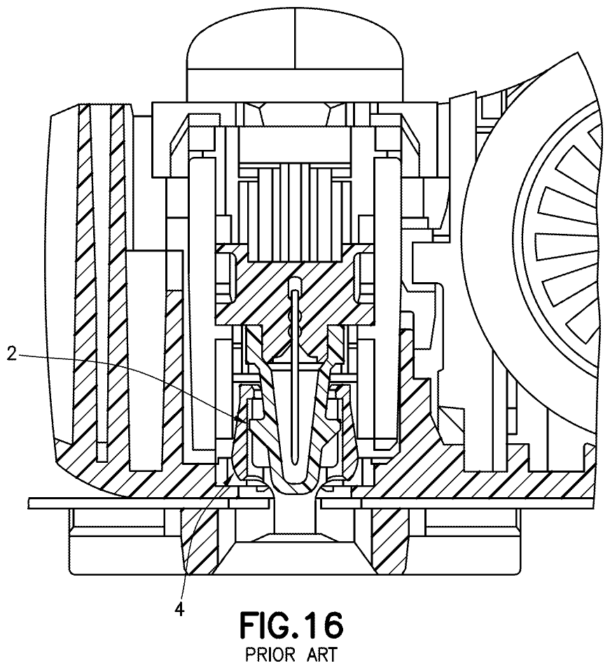

Referring to FIGS. 1-15, a wearable injector 10 according to one aspect of the present disclosure includes a drive assembly 12, a container 14, a valve assembly 16, and a needle actuator assembly 18. The drive assembly 12, the container 14, the valve assembly 16, and the needle actuator assembly 18 are at least partially positioned within a cavity defined by a housing 20. The housing 20 includes a top portion 22 and a bottom portion 24, although other suitable arrangements for the housing 20 may be utilized. In one aspect, the wearable injector 10 is an injector device configured to be worn or secured to a user and to deliver a predetermined dose of a medicament provided within the container 14 via injection into the user. The device 10 may be utilized to deliver a “bolus injection” where a medicament is delivered within a set time period. The medicament may be delivered over a time period of up to 45 minutes, although other suitable injection amounts and durations may be utilized. A bolus administration or delivery can be carried out with rate controlling or have no specific rate controlling. The device 10 may deliver the medicament at a fixed pressure to the user with the rate being variable. The general operation of the device 10 is described below in reference to FIGS. 1-15.

Referring again to FIGS. 1-15, the device 10 is configured to operate through the engagement of an actuation button 26 by a user, which results in a needle 28 of the needle actuator assembly 18 piercing the skin of a user, the actuation of the drive assembly 12 to place the needle 28 in fluid communication with the container 14 and to expel fluid or medicament from the container 14, and the withdrawal of the needle 28 after injection of the medicament is complete. The general operation of a wearable injector is shown and described in International Publication Nos. 2013/155153 and 2014/179774, which are hereby incorporated by reference in their entirety. The housing 20 of the device 10 includes an indicator window 30 for viewing an indicator arrangement 32 configured to provide an indication to a user on the status of the device 10 and a container window 31 for viewing the container 14. The indicator window 30 may be a magnifying lens for providing a clear view of the indicator arrangement 32. The indicator arrangement 32 moves along with the needle actuator assembly 18 during use of the device 10 to indicate a pre-use status, use status, and post-use status of the device 10. The indicator arrangement 32 provides visual indicia regarding the status, although other suitable indicia, such as auditory or tactile, may be provided as an alternative or additional indicia.

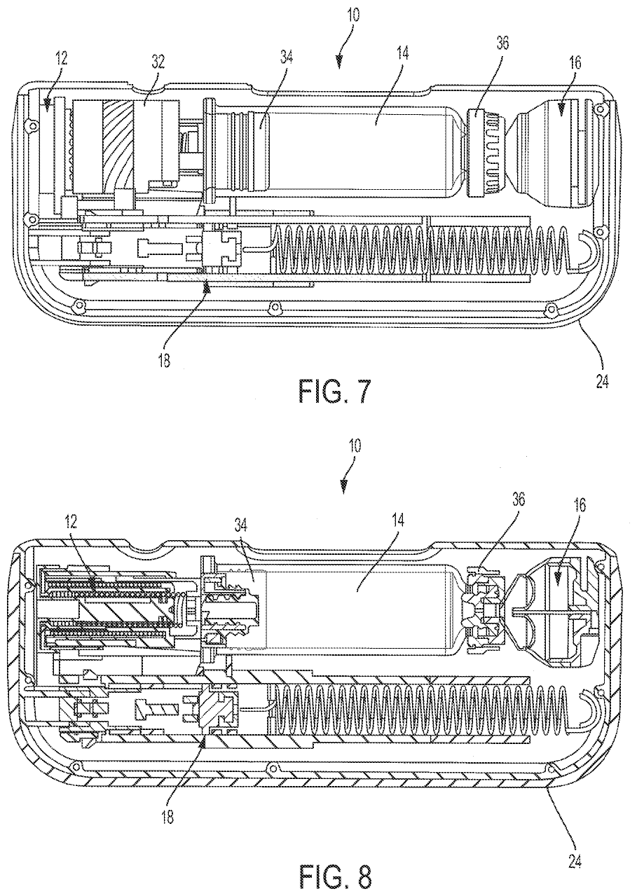

Referring to FIGS. 4-6, during a pre-use position of the device 10, the container 14 is spaced from the drive assembly 12 and the valve assembly 16 and the needle 28 is in a retracted position. During the initial actuation of the device 10, as shown in FIGS. 7-9, the drive assembly 12 engages the container 14 to move the container 14 toward the valve assembly 16, which is configured to pierce a closure 36 of the container 14 and place the medicament within the container 14 in fluid communication with the needle 28 via a tube (not shown) or other suitable arrangement. The drive assembly 12 is configured to engage a stopper 34 of the container 14, which will initially move the entire container 14 into engagement with the valve assembly 16 due to the incompressibility of the fluid or medicament within the container 14. The initial actuation of the device 10 is caused by engagement of the actuation button 26 by a user, which releases the needle actuator assembly 18 and the drive assembly 12 as discussed below in more detail. During the initial actuation, the needle 28 is still in the retracted position and about to move to the extended position to inject the user of the device 10.

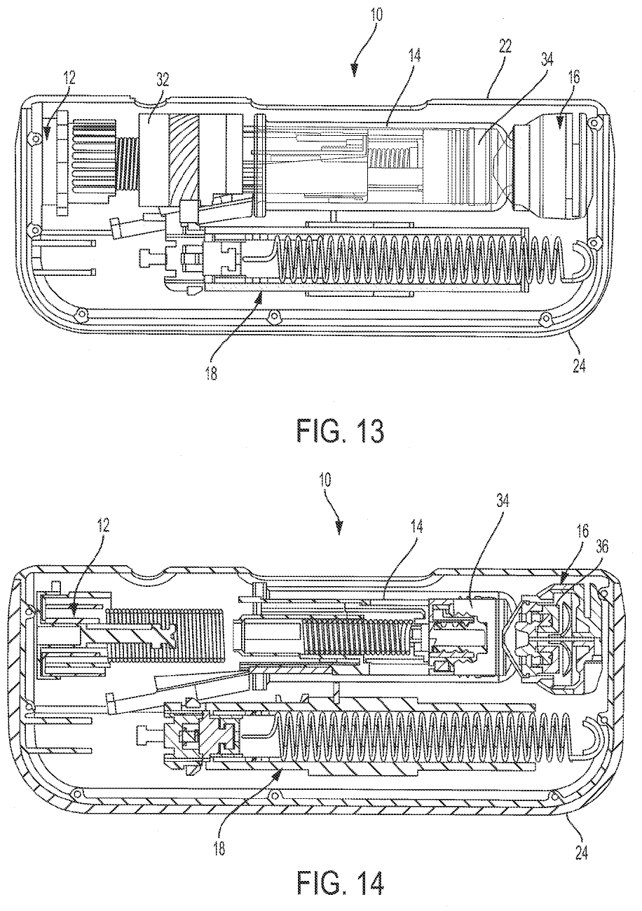

During the use position of the device 10, as shown in FIGS. 10-12, the needle 28 is in the extended position at least partially outside of the housing 20 with the drive assembly 12 moving the stopper 34 within the container 14 to deliver the medicament from the container 14, through the needle 28, and to the user. In the use position, the valve assembly 16 has already pierced a closure 36 of the container 14 to place the container 14 in fluid communication with the needle 28, which also allows the drive assembly 12 to move the stopper 34 relative to the container 14 since fluid is able to be dispensed from the container 14. At the post-use position of the device 10, shown in FIGS. 13-15, the needle 28 is in the retracted position and engaged with a pad 38 to seal the needle 28 and prevent any residual flow of fluid or medicament from the container 14. The container 14 and valve assembly 16 may be the container 14 and valve assembly 16 shown and described in International Publication No. WO 2015/081337, which is hereby incorporated by reference in its entirety.

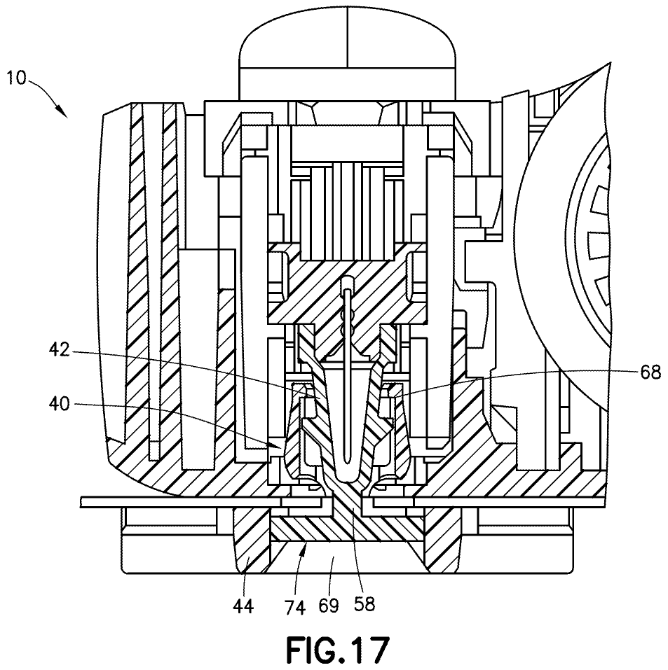

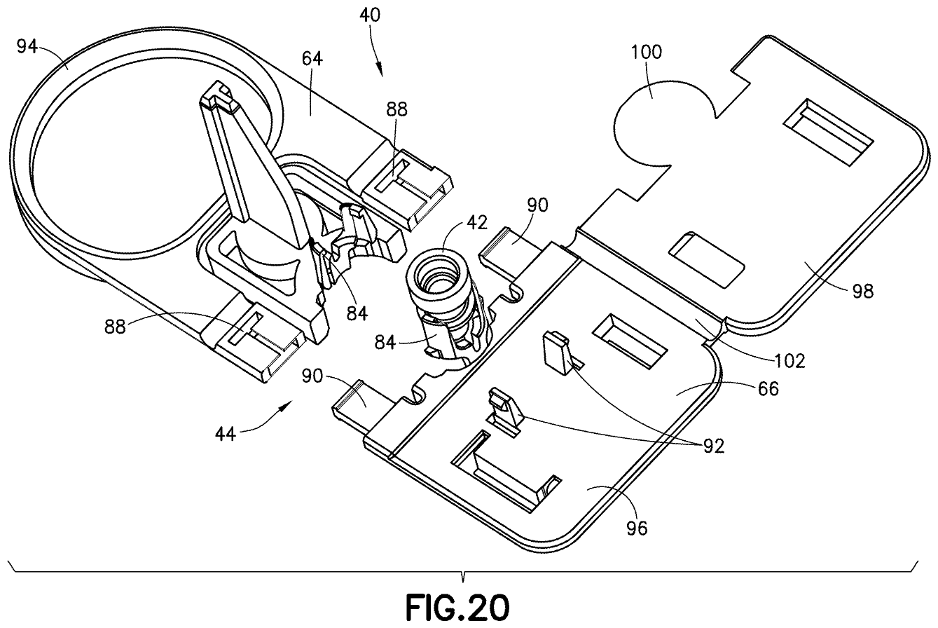

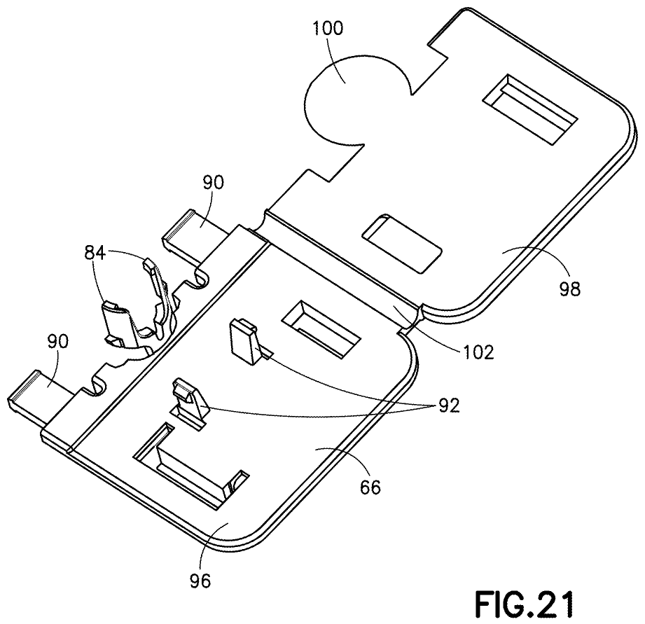

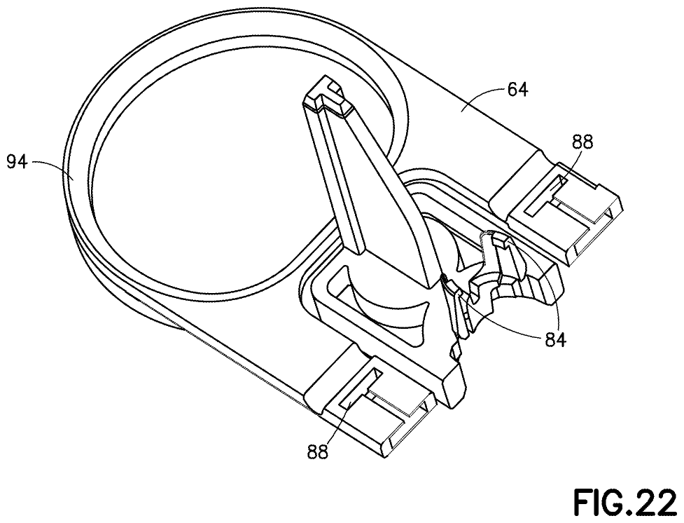

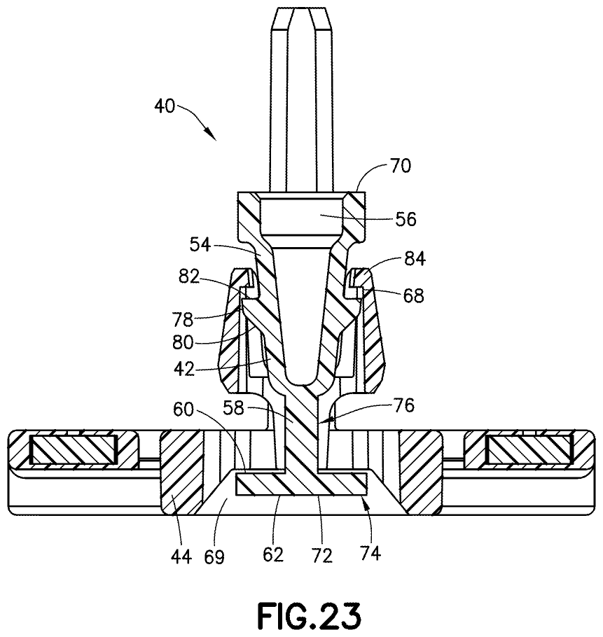

Referring to FIGS. 17-23, the wearable injector 10 of FIGS. 1-15 further includes a needle shield remover assembly 40 having a needle shield 42 and a needle shield remover 44. The needle shield 42 is configured to enclose at least a portion of the needle 28 of the wearable injector 10 to prevent accidental needle sticks and to maintain the sterility of the needle 28. As discussed in more detail below, separating the needle shield remover 44 from the wearable injector 10 also removes the needle shield 42 from the wearable injector 10. The needle shield remover assembly 40 may also include one or more features to prevent the actuation of the wearable injector 10 while the needle shield remover assembly 40 is connected to the wearable injector 10.

The needle shield 42 includes a body 54 defining an opening 56 configured to receive the needle 28 of the wearable injector 10. The body 54 of the needle shield 42 includes an extension portion 58 having a top surface 60 and bottom surface 62 positioned opposite the top surface 60. The needle shield remover 44 includes a first portion 64 and a second portion 66. The needle shield remover 44 has an unassembled position where the first portion 64 is spaced from the second portion 66 and an assembled position where the first portion 64 is connected to the second portion 66. The first portion 64 and the second portion 66 of the needle shield remover 44 defines a needle shield surface 68 configured to engage the top surface 60 of the extension portion 58 of the needle shield 42 when the needle shield remover 44 is in the assembled position. In one aspect or embodiment, the first portion 64 and the second portion 66 of the needle shield remover 44 together define a recessed portion 69 configured to receive at least a portion of the extension portion 58.

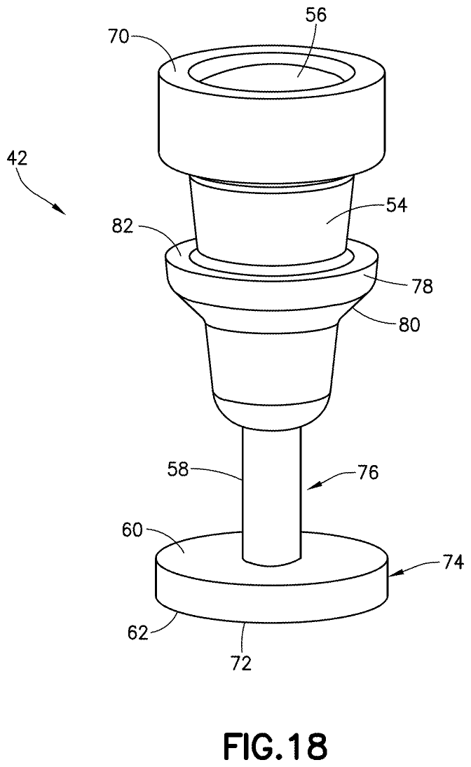

Referring to FIG. 18, the body 54 of the needle shield 42 has first end 70 and a second end 72 positioned opposite the first end 70. The opening 56 of the body 54 is positioned at the first end 70 and the extension portion 58 is positioned at the second end 72. The extension portion 58 includes a disc-shaped member 74, although other suitable shapes and arrangements may be utilized. The extension portion 58 also includes a cylindrical member 76, with the disc-shaped member 74 defining the second end 72 of the body 54 of the needle shield 42. The body 54 of the needle shield 42 includes a protrusion 78 having a tapered surface 80 and a planar surface 82, with the tapered surface 80 positioned closer to the second end 72 of the body 54 than the planar surface 82. As discussed below, the tapered surface 80 and the planar surface 82 interact with the needle shield remover 44 to allow the needle shield remover 44 to be connected to the needle shield 42 while still enabling the needle shield 42 to be removed by the needle shield remover 44 during use.

Referring again to FIGS. 17-23, the needle shield remover 44 includes a plurality of needle shield engagement members 84, with the plurality of needle shield engagement members 84 configured to be biased radially outward when engaged with the tapered surface 80 of the body 54 of the needle shield 42, and with the plurality of needle shield engagement members 84 configured to engage the planar surface 82 of the of needle shield 42 after the needle shield remover 44 is connected to the needle shield 42. With the needle shield 42 positioned over the needle 28 of the wearable injector 10, the plurality of needle shield engagement members 84 can be inserted into the wearable injector 10, with the needle shield remover 44 in the unassembled position, such that the needle shield engagement members 84 engage the tapered surface 80 of the protrusion 78 of the needle 28 to bias the needle shield engagement members 88 radially outward as the needle shield engagement members 84 are inserted into the housing 20 of the wearable injector 10. As an end of the needle shield engagement members 84 passes by the tapered surface 80 of the protrusion 78 of the needle shield 42, the needle shield engagement members 84 return to their original position. The needle shield remover 44 is then moved to the assembled position with the needle shield remover 44 connected to the housing 20 of the wearable injector 10. When the needle shield remover 44 is in the assembled positioned and connected to the wearable injector 10, subsequently removing the needle shield remover 44 from the housing 20 of the wearable injector 10, such as by pulling the needle shield 42 in a direction parallel to a longitudinal axis of the needle 28, will cause the needle shield surface 68 to engage the top surface 60 of the extension portion 58 of the needle shield 42 and/or cause the needle shield engagement members 84 to engage the planar surface 82 of the needle shield 42 to remove the needle shield 42 from the wearable injector 10. The first portion 64 and the second portion 66 of the needle shield remover 44 each include at least one of the plurality of needle shield engagement members 84, although other suitable arrangements may be utilized.

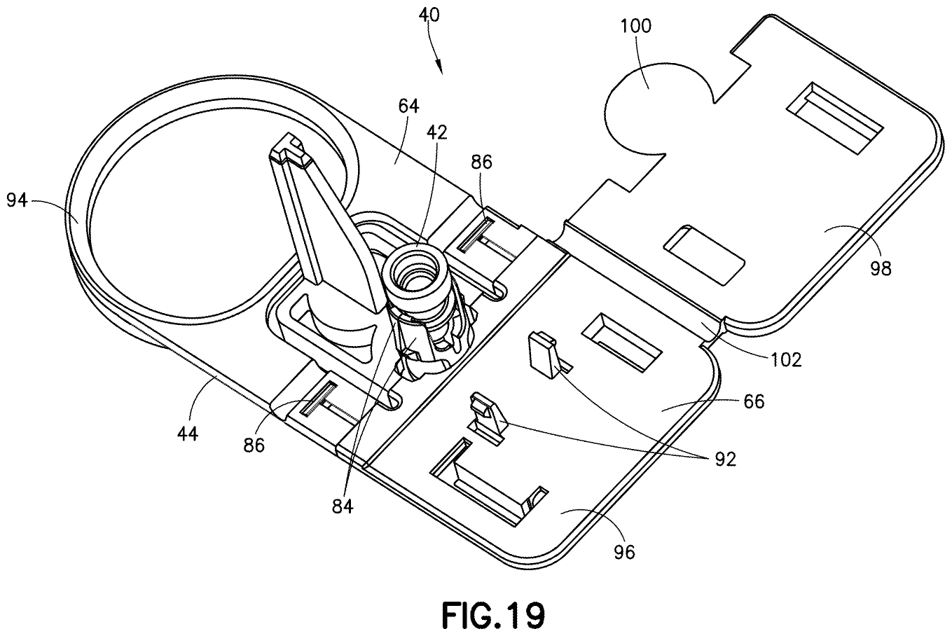

Referring to FIGS. 19-21, the first portion 64 of the needle shield remover 44 is connected to the second portion 66 of the needle shield remover 44 via a connector 86. The connector 86 includes a connector recess 88 positioned on one of the first portion 64 and the second portion 66 and a connector member 90 positioned on the other of the first portion 64 and the second portion 66. As shown in FIGS. 19-21, in one aspect or embodiment, the connector recess 88 is provided on the first portion 64 and the connector member 90 is provided on the second portion 66. The connector recess 88 and the connector member 90 form a snap fit connection, although other suitable arrangements may be utilized. The second portion 66 of the needle shield remover 44 includes a device connector 92 configured to secure the second portion 66 of the needle shield remover 44 to the wearable injector 10. The first portion 64 of the needle shield remover 44 includes a handle 94. The handle 94 may be grasped by a healthcare technician or patient to remove the needle shield remover 44 from the housing 20 of the wearable injector 10. The handle 94 is circular, although other suitable shapes and arrangements may be utilized. The second portion 66 of the needle shield remover 44 includes a first half 96 moveable relative to a second half 98. The second half 98 includes a cover 100 configured to cover the needle shield 42 after the needle shield remover 44 is connected to the needle shield 42. The first half 96 of the second portion 66 of the needle shield remover 44 is connected to the second half 98 of the second portion 66 of the needle shield remover 44 via a hinge 102, although other suitable arrangements may be utilized.

Elements of one disclosed aspect can be combined with elements of one or more other disclosed aspects to form different combinations, all of which are considered to be within the scope of the present invention.

While this disclosure has been described as having exemplary designs, the present disclosure can be further modified within the spirit and scope of this disclosure. This application is therefore intended to cover any variations, uses, or adaptations of the disclosure using its general principles. Further, this application is intended to cover such departures from the present disclosure as come within known or customary practice in the art to which this disclosure pertains and which fall within the limits of the appended claims.

1. A needle shield remover assembly for a wearable injector comprising:
a needle shield comprising a body defining an opening configured to receive a needle of the wearable injector, the body of the needle shield comprising an extension portion having a top surface and a bottom surface positioned opposite the top surface; and
a needle shield remover comprising a first portion and a second portion, the needle shield remover having an unassembled position where the first portion is spaced from the second portion and an assembled position where the first portion is connected to the second portion, the first portion and the second portion of the needle shield remover defining a needle shield surface configured to engage the top surface of the extension portion of the needle shield when the needle shield remover is in the assembled position.

2. The needle shield remover assembly of claim 1, wherein the body of the needle shield has a first end and a second end positioned opposite the first end, the opening of the body positioned at the first end and the extension portion positioned at the second end.

3. The needle shield remover assembly of claim 2, the extension portion comprising a disc-shaped member.

4. The needle shield remover assembly of claim 3, wherein the extension portion comprises a cylindrical member, with the disc-shaped member defining the second end of the body of the needle shield.

5. The needle shield remover assembly of claim 4, wherein the body of the needle shield comprises a protrusion having a tapered surface and a planar surface, the tapered surface positioned closer to the second end of the body than the planar surface.

6. The needle shield remover assembly of claim 5, wherein the needle shield remover comprises a plurality of needle shield engagement members, wherein the plurality of needle shield engagement members are configured to be biased radially outward when engaged with the tapered surface of the body of the needle shield, and wherein the plurality of needle shield engagement members are configured to engage the planar surface of the needle shield after the needle shield remover is connected to the needle shield.

7. The needle shield remover assembly of claim 6, wherein the first portion and the second portion of the needle shield remover each include at least one of the plurality of needle shield engagement members.

8. The needle shield remover assembly of claim 1, wherein the first portion of the needle shield remover is connected to the second portion of the needle shield remover via a connector.

9. The needle shield remover assembly of claim 8, wherein the connector comprises a connector recess positioned on one of the first portion and the second portion and a connector member positioned on the other of the first portion and the second portion.

10. The needle shield remover assembly of claim 9, wherein the connector recess and the connector member form a snap fit connection.

11. The needle shield remover assembly of claim 1, wherein the second portion of the needle shield remover comprises a device connector configured to secure the second portion of the needle shield remover to the wearable injector.

12. The needle shield remover assembly of claim 1, wherein the first portion of the needle shield remover comprises a handle.

13. The needle shield remover assembly of claim 1, wherein the second portion of the needle shield remover comprises a first half moveable relative to a second half, and wherein one of the first half and the second half comprises a cover configured to cover the needle shield after the needle shield remover is connected to the needle shield.

14. The needle shield remover assembly of claim 13, wherein the first half of the second portion of the needle shield remover is connected to the second half of the second portion of the needle shield remover via a hinge.

15. A wearable injector comprising:
a housing having a top surface and a bottom surface;
a container received within the housing, the container configured to receive a medicament, the container comprising a stopper configured to move within the container;
a drive assembly configured to move the stopper;
a needle having a retracted position where the needle is positioned within the housing and an extended position where a portion of the needle protrudes from the housing; and
a needle shield remover assembly comprising a needle shield comprising a body defining an opening configured to receive a needle of the wearable injector, the body of the needle shield comprising an extension portion having a top surface and a bottom surface positioned opposite the top surface, and
a needle shield remover comprising a first portion and a second portion, the needle shield remover having an unassembled position where the first portion is spaced from the second portion and an assembled position where the first portion is connected to the second portion, the first portion and the second portion of the needle shield remover defining a needle shield surface configured to engage the top surface of the extension portion of the needle shield when the needle shield remover is in the assembled position,
wherein the needle shield receives at least a portion of the needle, and wherein the needle shield remover is in the assembled position.

16. The wearable injector of claim 15, wherein the body of the needle shield has a first end and a second end position opposite the first end, the opening of the body positioned at the first end and the extension portion positioned at the second end.

17. The wearable injector of claim 16, the extension portion comprising a disc-shaped member.

18. The wearable injector of claim 17, wherein the extension portion comprises a cylindrical member, with the disc-shaped member defining the second end of the body of the needle shield.

19. The wearable injector of claim 18, wherein the body of the needle shield comprises a protrusion having a tapered surface and a planar surface, the tapered surface positioned closer to the second end of the body than the planar surface.

20. The wearable injector of claim 19, wherein the needle shield remover comprises a plurality of needle shield engagement members, wherein the plurality of needle shield engagement members are configured to be biased radially outward when engaged with the tapered surface of the body of the needle shield, and wherein the plurality of needle shield engagement members are configured to engage the planar surface of the needle shield after the needle shield remover is connected to the needle shield.
